(19) United States
(12) Patent Application Publication
(10) Pub. No.: US 2017/0000705 A1
(43) Pub. Date: Jan. 5, 2017

(54) COMPOSITION BASED ON A POLYUNSATURATED FATTY ACID AND ON A CAROTENOID, FOR ORAL ADMINISTRATION, FOR IMPROVING HAIR QUALITY

(71) Applicant: NUTRICOS TECHNOLOGIES, Clichy (FR)

(72) Inventor: Nathalie PICCARDI, Arceau (FR)

(73) Assignee: NUTRICOS TECHNOLOGIES, Clichy (FR)

(21) Appl. No.: 15/104,117

(22) PCT Filed: Dec. 11, 2014
(86) PCT No.: PCT/IB2014/066807

§ 371 Date: Jun. 13, 2016

(30) Foreign Application Priority Data
Dec. 12, 2013 (FR) 13 62462

(51) Int. Cl. A61K 8/36; A61K 8/92; A61K 8/67; A61K 8/97; A61K 8/31; A61Q 5/00; A61K 8/55; A61K 8/65; A61K 8/34; A61K 8/29; A61K 8/19; A61Q 7/00; A61K 8/11; A61K 8/37

(52) U.S. Cl. CPC A61K 8/361; A61K 8/11; A61K 8/925; A61K 8/922; A61K 8/676; A61K 8/678; A61K 8/97; A61K 8/31; A61K 8/375; A61K 8/553; A61K 8/65; A61K 8/345; A61K 8/29; A61K 8/19; A61Q 7/00; A61Q 5/00; A61K 2800/592

(57) ABSTRACT

The oral cosmetic use of a combination of active agents, including at least one polyunsaturated fatty acid and one carotenoid, for improving hair quality and more particularly hair quality at the level of a zone having grown before said use of a combination of said active agents. This combination of active agents may also include, by way of additional active agent, vitamin E, vitamin C or combinations thereof. The combination of active agents is preferably used in a cosmetic composition intended for oral administration, such as a food supplement.

### FIELD OF THE INVENTION

The invention relates to the oral cosmetic use of a combination of active agents based on a polyunsaturated fatty acid and on a carotenoid, for improving the quality of hair over its entire length.

### PRIOR ART

Hairs are produced in hair follicles formed from epithelial sheaths of epidermal origin and from a hair bulb containing bulb keratinocytes in a constant state of division during the hair growth phases. This growth is on average from 1 to 1.5 cm per month, i.e. 0.35 mm per day (Vogt A et al., Biology of the hair follicle, Hair growth and Disorders, published by Springer, pp 1-22). Each hair follicle follows its own hair cycle which consists of 3 phases. The follicles are programmed to perform 25 to 30 hair cycles during one's lifetime.

The possession of healthy, strong hair throughout one's lifetime is an ambition of most women and men.

To this effect, there are numerous shampoo, conditioner, mask, hair-oil formulations, etc., which act directly on the hair or the scalp. Orally ingested food supplements are also used for hair care, for example for reducing hair loss or promoting hair growth. The scalp comprises 100 000 to 150 000 hairs. The dermal papilla provides the nutritive elements required for the formation of keratin which is essential for hair growth. The existing food supplements therefore act at the base of the hair.

The food supplements available on the market are predominantly based on vitamin B and sometimes contain vitamin A, vitamin E or vitamin C. In addition to these vitamins, food supplements generally contain sulfur-containing amino acids and/or zinc.

Vitamins B5 and B8 are, for example, used to prevent hair loss, vitamins B2 and B6 to prevent and treat dandruff and vitamin B3 to stimulate the microcirculation. Moreover, sulfur-containing amino acids, such as methionine and cysteine, are used to promote hair growth. However, all of these compounds act on the hair which is growing during the taking of the food compound.

There is therefore a need to obtain food supplements which improve the quality of hair over its entire length, that is to say also the hair and the hair shaft which have been formed well before the taking of the food supplement.

The aim of the present invention is to satisfy this need.

Indeed, the inventors have demonstrated, surprisingly, that after 3 months and 6 months of oral administration of at least one carotenoid, of vitamin C, of vitamin E and of at least one polyunsaturated fatty acid, in particular originating from fish oil and/or from blackcurrant seed oil, hair quality is improved on the zone of the hair located 6 to 10 cm from the scalp, that is to say the zone of the hair having already grown approximately 6 to 10 months before the taking of the food supplement.

### SUMMARY OF THE INVENTION

The present invention is consequently based on the demonstration by the inventors that the oral administration of at least one carotenoid, of vitamin C, of vitamin E and of at least one polyunsaturated fatty acid, in particular originating from fish oil and/or from blackcurrant seed oil, makes it possible to improve hair quality and more particularly hair quality at the level of a zone having grown before the oral administration of at least one carotenoid, of vitamin C, of vitamin E and of at least one polyunsaturated fatty acid, in particular originating from fish oil and/or from blackcurrant seed oil.

Thus, surprisingly, the inventors have demonstrated that cosmetic compositions intended for oral administration are of use for improving hair quality, and more particularly for increasing the fibre diameter and/or the fibre density and/or increasing hair volume and/or improving the quality of the hair fibre, at the level of a zone having grown before the oral administration of at least one carotenoid, of vitamin C, of vitamin E and of at least one polyunsaturated fatty acid, in particular originating from fish oil and/or from blackcurrant seed oil.

The inventors have in particular used at least one cosmetic composition which can be in the form of soft capsules and which comprises a combination of active agents comprising polyunsaturated fatty acids present in the form of fish oil and of blackcurrant seed oil, a carotenoid, vitamin C and vitamin E.

The administration of the composition above, in particular at a rate of two capsules per day, makes it possible to improve hair quality and more particularly hair quality at the level of a zone having grown before the administration of said composition.

A subject of the invention is thus the oral cosmetic use of a combination of active agents comprising at least one polyunsaturated fatty acid, vitamin C, vitamin E and a carotenoid, for improving hair quality, and more particularly for increasing the fibre diameter and/or the fibre density and/or increasing hair volume and/or improving the quality of the hair fibre, at the level of a zone having grown before the oral administration of at least one carotenoid, of vitamin C, of vitamin E and of at least one polyunsaturated fatty acid, in particular originating from fish oil and/or from blackcurrant seed oil, in particular of men and women.

A subject of the invention is particularly the cosmetic use above, in which the combination of active agents is used in a cosmetic composition suitable for oral administration.

A subject of the invention is also a cosmetic composition intended for oral administration, comprising:

(i) at least one polyunsaturated fatty acid in a content of between 1% and 80% by weight, relative to the total weight of the cosmetic composition,

(ii) at least one carotenoid in a content of between 0.01% and 6% by weight, relative to the total weight of the composition,

(iii) optionally vitamin E in a content of between 0.001% and 10% by weight, relative to the total weight of the composition, and

(iv) optionally vitamin C in a content of between 0.001% and 30% by weight, relative to the total weight of the composition.

Kit

A subject of the present invention is a cosmetic combination product or kit for use simultaneously, separately or spread out over time, comprising a first cosmetic composition comprising a part of the compounds forming the combination of active agents, as defined above, and a second cosmetic composition comprising at least the other part of the compounds forming said combination of active agents.

Process

The present invention also relates to a cosmetic process for improving hair quality and more particularly hair quality at the level of a zone having grown before the oral administration of a combination of active agents, as defined above, comprising the oral administration, to an individual, of said combination of active agents, of a cosmetic composition intended for oral administration as defined above, or of a cosmetic combination product or kit as defined above.

### DETAILED DESCRIPTION

The main subject of the invention is therefore the oral cosmetic use of a combination of active agents comprising at least one carotenoid and at least one polyunsaturated fatty acid, in particular originating from fish oil and/or from blackcurrant seed oil, for improving hair quality and more particularly for improving hair quality at the level of a zone having grown before the oral administration of the active agents, in particular in men or in women.

Preferably according to the invention, the oral cosmetic use of a combination of active agents comprising at least one carotenoid and at least one polyunsaturated fatty acid, in particular originating from fish oil and/or from blackcurrant seed oil, is characterized in that the improvement of hair quality at the level of a zone having grown before said use comprises an increased fibre diameter and/or fibre density and/or an improved hair volume and/or an improved quality of the hair fibre at the level of a zone having grown before the oral administration of the abovementioned composition.

According to a particular embodiment of the present invention, the improvement of hair quality consists in an increased fibre diameter and/or an increased fibre density and/or an improved quality of the hair fibre, and more preferably in an increased fibre diameter and/or an increased fibre density.

The term “at the level of a zone having grown before” is intended to denote, according to the invention, the hair having grown before the day of the first oral administration of the combination of active agents as defined according to the invention. It is known that the hair grows approximately 1 cm every month; after 3 months, this zone is therefore defined on the fibre: at 3 centimetres from the scalp up to the end of the hair and preferentially from 3 to 10 centimetres from the scalp. After 6 months, this zone is defined on the fibre at 6 centimetres from the scalp up to the end of the hair and preferentially from 6 to 10 centimetres from the scalp, and even more preferentially it is at 10 centimetres from the scalp.

The term “hair over its entire length” is intended to denote, according to the invention, ranging from the scalp up to the end.

The term “increasing the density” is intended to mean, according to the invention, a larger number of hairs per cm². The hair density can also be measured with Trichoscan®. With this apparatus, a density of less than 250 hairs per cm² is considered to be a low density. A 5% increase in the density can be considered to be clinically significant and visible. The term “increasing hair fibre diameter” is intended to denote, according to the invention, an increase in the number of hairs having a diameter greater than 40 μm. A 5% increase in the number of hairs with a diameter greater than 40 μm can be considered to be significant.

The density and the diameter can also be evaluated by measuring the cross section of a lock of hair using an apparatus called a Trichometer [www.haircheck.com; Cohen B. The cross section trichometer: a new device for measuring hair quantity, hair loss and hair growth, Dermatol Surg, 2008; 34: 900-911].

The term “increasing hair volume” is intended in particular to denote, according to the invention, an increase in the diameter of the hairs, associated with a decrease in the heterogeneity of the diameter of the hairs, and with an increase in the density.

The heterogeneity of the hair diameter can be measured by means of a binocular magnifying lens, for example by sorting a lock of 50 to 100 cut (or well removed) hairs, by categorizing them, for example into four categories (very thin, thin, thick and very thick) and by calculating the percentage of hairs in each category. The heterogeneity of the hair diameter is then evaluated by comparing the change in these percentages over time.

The term “improving the quality of the hair fibre” is intended to denote an increase in the tensile strength of the hair, and/or the preventing and/or combatting of brittle hair.

The term “preventing and/or combatting brittle hair” is intended to denote, according to the invention, an overall improvement in the structure of the hair shaft and in particular of the cuticle, the outermost layer of the hair. The quality of the cuticle can be evaluated, for example by means of a microscope, by noting the presence of tightened or loose scales, of intact or non-intact scales and/or of broken, or even missing, scales. A 5% decrease in the number of loose, broken or missing scales can be considered to be significant.

The term “improving the tensile strength of the hair” is intended to denote, according to the invention, the solidity of the hair, which can be measured by the tensile test. This measurement for determining the tensile mechanical properties of the hair can be carried out using a commercial tool, the MTT600 (mini Tensile Tester) from the company Dia Stron (http://www.diastron.com/). A 5% increase in the force required in order to break the hair is considered to be significant.

The various parameters as defined above can be evaluated by means of a multiple-choice questionnaire filled out by men and/or women in the context of an observational study which can be carried out at dermatologist practices. An effect will be considered to be significant starting from when at least 50% of men and/or women have perceived a positive effect.

Polyunsaturated Fatty Acids

For the purposes of the present invention, the term “polyunsaturated fatty acid” is intended to mean a fatty acid comprising at least two double bonds. This involves more particularly long-chain fatty acids, i.e. having at least 14 carbon atoms.

The polyunsaturated fatty acids may be in acid form, in the form of triglycerides or in the form of methyl or ethyl esters.

The polyunsaturated fatty acids comprise in particular ω-3 fatty acids, ω-6 fatty acids and ω-9 fatty acids, characterized by the position of the unsaturation closest to the end methyl group, and mixtures thereof.

The terms “w” and “omega” are subsequently used without distinction. Polyunsaturated fatty acids comprising from 18 to 22 carbon atoms, more preferentially ω-3, ω-6 and ω-9 fatty acids, are quite particularly suitable for the invention. The omega 6 and omega 3 polyunsaturated fatty acids may be present in the composition at a ratio between 1/600 and 600/1, in particular between 1/400 and 400/1 and preferably between 1/300 and 300/1.

Among the polyunsaturated fatty acids of the ω-6 series, mention may in particular be made of linoleic acid comprising 18 carbon atoms and two unsaturations or LA (18:2, ω-6), γ-linolenic acid comprising 18 carbon atoms and three unsaturations or GLA (18:3, ω-6), di-homogamalinolenic acid comprising 20 carbon atoms and 3 unsaturations (20:3, ω-6), arachidonic acid, 5,8,11,14-eicosatetraenoic acid (20:4, ω-6) and docosatetraenoic acid (22:4, ω-6).

The polyunsaturated fatty acids of the ω-3 series can in particular be chosen from α-linolenic acid or ALA (18:3, ω-3), stearidonic acid or SDA (18:4, ω-3), 5,8,11,14,17-eicosapentaenoic acid or EPA (20:5, ω-3), 4,7,10,13,16,19-docosahexaenoic acid or DHA (22:6, ω-3), docosapentanoic acid or DPA (22:5, ω-3) and n-butyl-5,11,14-eicosatrienonic acid.

The polyunsaturated fatty acids of the ω-9 series can in particular be chosen from oleic acid (18:1, ω-9) and erucic acid (22:1, ω-9).

α-Linolenic acid, linoleic acid, γ-linolenic acid, stearidonic acid, eicosapentaenoic acid, docosahexaenoic acid, oleic acid, an extract comprising them, or combinations thereof, are quite particularly suitable for the invention.

According to one variant of the invention, the polyunsaturated fatty acid(s) considered is (are) used in an isolated form, i.e. after extraction from its (their) source(s) of origin.

The total content of polyunsaturated fatty acid(s) in a composition in accordance with the invention can range from 1% to 80% by weight, preferably from 5% to 70% by weight, more preferentially from 10% to 60% by weight and even more preferentially from 20% to 50% by weight, relative to the total weight of the composition.

For example, a composition in accordance with the invention can comprise a total content of polyunsaturated fatty acid(s) of between 35% and 45% by weight.

A composition in accordance with the invention, as developed hereinafter, can comprise the polyunsaturated fatty acid(s) in a concentration adjusted so that the polyunsaturated fatty acid(s) is (are) administered at a content ranging from 100 mg/day to 1500 mg/day, preferably from 200 mg/day to 700 mg/day and more preferentially from 350 mg/day to 550 mg/day.

For example, a composition in accordance with the invention can comprise the polyunsaturated fatty acid(s) in a concentration adjusted so that the polyunsaturated fatty acid(s) is (are) administered at a content ranging from 400 mg/day the 500 mg/day.

The source of polyunsaturated fatty acid can be chosen from plant oils, for instance evening primrose oil, borage oil, blackcurrant seed oil, Echium oil and hemp oil, and an extract of the microalga Schizochytrium sp, or combinations thereof. The plant source of polyunsaturated fatty acid is preferably blackcurrant seed oil.

The source of polyunsaturated fatty acid can also be chosen from fish oils.

Plant oils from walnut, hazelnut, almond (Juglans regia), coriander, soya bean (Glycina max), rapeseed (Brassica naptus), chia, and linseed and fish oils, for example, are rich in polyunsaturated fatty acids of the ω-3 series.

The ω-3 polyunsaturated fatty acids can also be found in zooplankton, crustaceans/molluscs and fish. Fish oils constitute the main industrial source of EPA and DHA. Microalgal biomasses can also constitute a raw material for extraction of ω-3 polyunsaturated fatty acids.

Thus, a polyunsaturated fatty acid can be used in a combination of active agents in accordance with the invention in the form of at least one oil chosen from evening primrose oil, borage oil, blackcurrant seed oil, walnut oil, soya bean oil, fish oil, sunflower oil, wheat germ oil, hemp oil, fenugreek oil, Echium oil, argan oil, rice bran oil, sesame oil, almond oil, hazelnut oil, chia oil, linseed oil, olive oil, avocado oil, safflower oil, camelina oil, Inca Inchi oil, passionfruit oil, rasberry seed oil, cranberry seed oil, blueberry seed oil, Rubus chamaemorus Linn. oil, sea buckthorn oil, cumin oil, kiwi seed oil, coriander oil, microalgal extract oil, zooplankton extract oil, crustacean and/or mollusc extract oil, or combinations thereof.

The microalgal extract oil is, for example, a Schizochytrium sp extract oil.

The oil is preferably a blackcurrant seed oil and/or a fish oil, more preferentially a blackcurrant seed oil and a fish oil.

A composition in accordance with the invention, as developed hereinafter, can comprise an oil and/or an extract and/or a biomass in a content of between 20% and 99% by weight, preferably between 30% and 95% by weight, more preferentially between 50% and 90% and even more preferentially between 60% and 85%, relative to the total weight of the composition.

For example, a composition in accordance with the invention can comprise an oil and/or an extract and/or a biomass in a content of between 80% and 85% by weight, relative to the total weight of the composition.

A composition in accordance with the invention, as developed hereinafter, can comprise an oil and/or an extract and/or a biomass in a concentration adjusted so that said oil and/or extract and/or biomass is administered at a content ranging from 100 mg/day to 5000 mg/day, preferably from 150 mg/day to 3000 mg/day and more preferentially from 200 mg/day to 1000 mg/day.

For example, a composition in accordance with the invention can comprise an oil and/or an extract and/or a biomass in a concentration adjusted so that said oil and/or extract and/or biomass is administered at a content ranging from a 200 mg/day to 500 mg/day.

Thus, according to one preferred embodiment, a combination of active agents in accordance with the invention comprises at least one oil chosen from a blackcurrant seed oil and/or a fish oil.

According to a further preferred embodiment, a combination of active agents in accordance with the invention comprises a blackcurrant seed oil and a fish oil, preferably in a content of between 80% and 85% by weight, for example for an administration at a content ranging from 200 mg/day to 500 mg/day.

Additional Active Agents

A combination of active agents in accordance with the invention can also comprise one or more other additional cosmetic active agent(s).

Advantageously, such an additional cosmetic active agent may be intended to reinforce the desired cosmetic effect as previously described.

Needless to say, those skilled in the art will take care to select the additional active agents and the amount thereof such that the advantageous properties of the composition in accordance with the invention are not, or are not substantially, adversely affected by the envisaged addition.

By way of additional active agent that can be used, mention may be made of:
vitamins other than vitamin E, such as vitamin A, or at least one B-group vitamin, for example vitamin B1 (also called thiamine), vitamin B2 (also called riboflavin), vitamin B3 (also called niacin or vitamin PP), vitamin B5, vitamin B6, vitamin B8, vitamin B9 and vitamin B12,
antioxidants, such as curcuminoids; carotenoids, in particular chosen from β-carotene, astaxanthin, zeaxanthin, lutein or compounds containing them, such as goji berries; polyphenol compounds, flavonoids such as catechins; proanthocyanidins, anthocyanins, OPCs (oligomeric proanthocyanidins); ubiquinones; coffee extracts containing polyphenols and/or diterpenes; chicory extracts; ginkgo biloba extracts; proanthocyanidin-rich grape extracts; capsicum extracts; soya bean extracts; cocoa; pomegranate; Emblica; coenzyme Q10; selenium,
minerals, calcium, magnesium, copper, iron, iodine, manganese, selenium, chromium(III),
sugars,
amino acids, in particular sulfur-containing amino acids, such as glutathione precursors, selenium amino acids, citrulline,
phytosterols,
resveratrol,
hesperidin, neohesperidin,
orthosilicic acid, monomethylsilanetriol, and
mixtures thereof.

In another particularly advantageous embodiment of the invention, the combination of active agents comprises, by way of additional active agent, at least one compound chosen from vitamin C, vitamin E or a carotenoid.

The combination of active agents can, for example, comprise, by way of additional active agent, at least two compounds chosen from vitamin C, vitamin E or a carotenoid, or else these three compounds.

Vitamins C and E

According to yet another of its aspects, the present invention relates to a combination of active agents comprising at least one polyunsaturated fatty acid and at least one other vitamin chosen from vitamin C and vitamin E.

The vitamin E is preferably in the form of tocopheryl acetate.

According to yet another of its aspects, the present invention relates to a combination of active agents comprising at least one fish oil comprising at least one polyunsaturated fatty acid, vitamin C and vitamin E. Preferably, a combination of active agents in accordance with the invention comprises at least one fish oil comprising at least one polyunsaturated fatty acid, vitamin C, and also vitamin E.

According to yet another of its aspects, the present invention relates to a combination of active agents comprising at least one blackcurrant seed oil comprising at least one polyunsaturated fatty acid, vitamin C and vitamin E. Preferably, a combination of active agents in accordance with the invention comprises at least one blackcurrant seed oil comprising at least one polyunsaturated fatty acid, vitamin C, and also vitamin E.

According to yet another of its aspects, the present invention relates to a combination of active agents comprising at least one blackcurrant seed oil and one fish oil, vitamin E and vitamin C.

A composition according to the invention, as developed hereinafter, can comprise vitamin C in a content of between 0.001% and 30% by weight, preferably between 0.01% and 25% by weight, more preferentially between 0.1% and 20% by weight and even more preferentially between 1% and 15% by weight, relative to the total weight of the composition.

For example, a composition in accordance with the invention can comprise vitamin C in a content of between 2% and 3% by weight, relative to the total weight of the composition.

A composition in accordance with the invention, as developed hereinafter, can comprise vitamin C in a concentration adjusted so that it is administered at a content ranging from 5 mg/day to 100 mg/day, preferably from 10 mg/day to 90 mg/day and more preferentially from 20 mg/day to 80 mg/day.

For example, a composition in accordance with the invention can comprise vitamin C in a concentration adjusted so that it is administered at a content ranging from 25 mg/day to 30 mg/day.

A composition according to the invention, as developed hereinafter, can comprise vitamin E in a content of between 0.001% and 10% by weight, preferably between 0.01% and 10% by weight, more preferentially between 0.1% and 5% by weight and even more preferentially between 0.2% and 2% by weight, relative to the total weight of the composition.

For example, a composition in accordance with the invention can comprise vitamin E in a content of between 0.4% and 0.5% by weight, relative to the total weight of the composition.

A composition in accordance with the invention, as developed hereinafter, can comprise vitamin E in a concentration adjusted so that it is administered at a content ranging from 0.1 mg/day to 20 mg/day, preferably from 1 mg/day to 15 mg/day and more preferentially from 2 mg/day to 12 mg/day.

For example, a composition in accordance with the invention can comprise vitamin E in a concentration adjusted so that it is administered at a content ranging from 4 mg/day to 6 mg/day.

Carotenoid

A preferred combination of active agents according to the invention comprises at least one carotenoid.

In the context of the present invention, the term “carotenoid” is intended to mean both a carotenoid with provitamin A activity and a carotenoid with no provitamin A activity.

Needless to say, according to the invention, the carotenoid may be a mixture of carotenoids with provitamin A activity and of carotenoids with no provitamin A activity. This mixture may be in any proportion.

According to the invention, the carotenoid with provitamin A activity may be a mixture of carotenoids with provitamin A activity. This mixture may be in any proportion. Among the carotenoids with provitamin A activity, examples that may be mentioned include β-carotene and α-carotene, preferably β-carotene.

According to the invention, the carotenoid with no provitamin A activity may be a mixture of carotenoids with no provitamin A activity. This mixture may be in any proportion. Among the carotenoids with no provitamin A activity, examples that may be mentioned include zeaxanthin, cryptoxanthin, lutein and lycopene.

More particularly, the carotenoid used in the context of the present invention is lycopene.

In other words, the carotenoid present in the combination of active agents of the present invention comprises, or even consists of, lycopene.

A carotenoid in accordance with the invention may also be astaxanthin.

The carotenoid used according to the invention may be of natural or synthetic origin. The term “natural origin” refers to a carotenoid, in pure form or in solution irrespective of its concentration in said solution, obtained from a natural element such as a plant extract. For example, when the carotenoid is lycopene, a tomato extract can more particularly be used.

Thus, according to one preferred embodiment of the invention, the carotenoid used in a combination according to the invention is lycopene, preferably in a tomato extract, more preferentially a lycopene-rich tomato extract.

A lycopene-rich tomato extract comprises, for example, at least 5% of lycopene, more preferentially at least 7% of lycopene, even more preferentially at least 10% of lycopene by weight, relative to the total weight of the extract.

A lycopene-rich tomato extract advantageously comprises at least 10% of lycopene by weight, relative to the total weight of the extract.

The term “synthetic origin” is intended to mean lycopene, in pure form or in solution irrespective of its concentration in said solution, obtained via chemical synthesis. The lycopene which can be used in the context of the present invention may be in cis or trans chemical form.

When the carotenoid is of natural origin, it may be obtained from a plant material derived from the whole plant cultivated in vivo or derived from in vitro culture.

The term “cultivated in vivo” is intended to mean any cultivation of standard type, i.e. in soil in the open air or in a greenhouse, or alternatively without soil.

The term “in vitro culture” is intended to mean all the techniques known to those skilled in the art which make it possible to artificially obtain a plant or a plant part. The selection pressure imposed by the physicochemical conditions during the growth of plant cells in vitro makes it possible to obtain a standardized plant material that is available throughout the year, in contrast with plants cultivated in vivo.

Preferentially, according to the invention, a plant derived from in vivo cultivation is used. Any extraction method known to those skilled in the art may be used to prepare the carotenoid used according to the invention.

Very preferentially, in the case of lycopene, a lycopene-rich tomato extract is used.

Lycopene is also present in melon, guava and grapefruit.

The lycopene may be in alcoholic solution, in particular ethanolic solution. The carotenoid may also be in lipid or lipoalcoholic solution.

The lycopene may be in aqueous suspension. For this, use may be made of forms which are water-dispersible, under cold or hot conditions.

Any other more complex lycopene-based ingredient may also be used for implementing the invention.

Thus, a “more complex ingredient” is intended to mean, for example, a primary composition comprising lycopene and a whey protein. This primary composition is in particular described in document WO 01/91588. This primary composition is also known as lactolycopene. It has the advantage of increasing the bioavailability of the lycopene and/or of being easy to formulate in food supplements (sachet, gel capsule, tablet, sugar-coated tablet, soft capsule, etc., forms).

The lactolycopene may in particular be sold by the company Indena.

The amount of extract that may be used according to the invention obviously depends on the desired effect, and may thus vary within a wide range.

The total content of carotenoid(s), preferably of lycopene, in a composition in accordance with the invention can range from 0.01% to 6% by weight, preferably from 0.02% to 4% by weight, more preferentially from 0.05% to 3% by weight and even more preferentially from 0.07% to 2% by weight, relative to the total weight of the composition.

For example, a composition in accordance with the invention can comprise a total content of carotenoid(s), preferably of lycopene, of between 0.09% and 0.1% by weight, relative to the total weight of the composition.

A composition in accordance with the invention, as developed hereinafter, can comprise at least one carotenoid, preferably lycopene, in a total concentration of carotenoid(s) adjusted for administration at a content ranging from 0.1 mg/day to 10 mg/day, preferably from 0.5 mg/day to 8 mg/day and more preferentially from 0.8 mg/day to 6 mg/day.

For example, a composition in accordance with the invention can comprise at least one carotenoid, preferably lycopene, in a concentration adjusted so that it is administered at a content ranging from 1 mg/day to 1.5 mg/day.

Thus, a combination of active agents in accordance with the invention advantageously comprises at least one oil comprising at least one polyunsaturated fatty acid, and isolated lycopene and/or a lycopene-rich extract.

According to one preferred embodiment, a combination of active agents in accordance with the invention comprises fish oil and/or blackcurrant seed oil, and a tomato extract.

According to a further preferred embodiment, a combination of active agents in accordance with the invention comprises fish oil, blackcurrant seed oil, vitamin E and a tomato extract.

Preferably, a combination of active agents in accordance with the invention comprises, in addition to at least one polyunsaturated fatty acid, lycopene, vitamin C and/or vitamin E, preferentially vitamin C and vitamin E.

Antioxidants

A combination of active agents in accordance with the invention may comprise, in addition to at least one polyunsaturated fatty acid and a carotenoid, an antioxidant other than carotenoids.

Such antioxidants are known to those skilled in the art.

The antioxidants may, for example, be chosen from curcuminoids; polyphenol compounds, flavonoids such as catechins; proanthocyanidins, anthocyanins, OPCs (oligomeric proanthocyanidins); ubiquinones; coffee extracts containing polyphenols and/or diterpenes; chicory extracts; ginkgo biloba extracts; proanthocyanidin-rich grape extracts; capsicum extracts; soya bean extracts; cocoa; pomegranate; Emblica; coenzyme Q10; selenium.

Anti-Inflammatory Agent

A combination of active agents in accordance with the invention may comprise, in addition to at least one polyunsaturated fatty acid, an anti-inflammatory agent.

The anti-inflammatory agents are known to those skilled in the art.

An anti-inflammatory agent in accordance with the invention may be chosen from flavonoids such as catechins, proanthocyanidins, anthocyanins, OPCs (oligomeric proanthocyanidins), and flavonones, for example hesperidin.

In one particular embodiment, the combination of active agents does not comprise an anti-inflammatory agent.

Probiotic and Prebiotic Microorganism

According to one particular embodiment, a composition of the invention may also comprise at least one probiotic microorganism, at least one prebiotic, or combinations thereof.

Specific examples of probiotic microorganisms that are suitable for the invention are microorganisms of the genus Bifidobacterium, such as Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (LC1, NCFB 1748); microorganisms of the genus Lactobacillus, such as Lactobacillus amylovorus, Lactobacillus casei (Shirota), Lactobacillus rhamnosus (strain GG), Lactobacillus brevis, Lactobacillus crispatus, Lactobacillus delbrueckii (subsp. bulgaricus, lactis), Lactobacillus fermentum, Lactobacillus helveticus, Lactobacillus gallinarum, Lactobacillus gasseri, Lactobacillus johnsonii, Lactobacillus paracasei, Lactobacillus plantarum, Lactobacillus rhamnosus, Lactobacillus salivarius, Lactobacillus alimentarius, Lactobacillus curvatus, Lactobacillus casei subsp. casei, Lactobacillus sake, microorganisms of the genus Lactococcus, such as Lactococcus lactis, Lactococcus lactis subspp. lactis or cremoris, microorganisms of the genus Enterococcus, such as Enterococcus faecalis or Enterococcus faecium, microorganisms of the genus Leuconostoc, such as Leuconostoc mesenteroides subsp. dextranicum, microorganisms of the genus Pediococcus, such as Pediococcus acidilactici, microorganisms of the genus Sporolactobacillus, such as Sporolactobacillus inulinus, microorganisms of the genus Streptococcus, such as Streptococcus salvarius subsp. thermophilus, Streptococcus thermophilus, microorganisms of the genus Staphylococccus, such as Staphylococccus carnosus, Staphylococcus xylosus, microorganisms of the genus Saccharomyces, such as Saccharomyces cerevisiae or else Saccharomyces boulardii, microorganisms of the genus Bacillus, such as Bacillus cereus var. toyo, Bacillus subtilis, Bacillus coagulans, Bacillus licheniformis, microorganisms of the genus Escherichia, such as Escherichia coli strain nissle, microorganisms of the genus Propionibacterium, such as Propionibacterium freudenreichii, or combinations thereof.

The microorganisms may be formulated in the form of powders, i.e. in a dry form, or in the form of suspensions or solutions.

More particularly, the microorganism may be a probiotic microorganism chosen from microorganisms of the genus Lactobacillus sp. and/or Bifidobacterium sp., a fraction thereof and/or a metabolite thereof. As illustrations of these microorganisms, mention may be made more particularly of Lactobacillus johnsonii, Lactobacillus rhamnosus, Lactobacillus paracasei, Lactobacillus casei, Bifidobacterium bifidum, Bifidobacterium breve, Bifidobacterium longum, Bifidobacterium animalis, Bifidobacterium lactis, Bifidobacterium infantis, Bifidobacterium adolescentis and Bifidobacterium pseudocatenulatum, or combinations thereof.

The species that are quite particularly suitable are Lactobacillus johnsonii, Lactobacillus paracasei, Bifidobacterium adolescentis, Bifidobacterium longum or Bifidobacterium lactis NCC 2818 (also denoted Bb12 ATCC 27536), which were deposited, respectively, according to the Treaty of Budapest, at the Institut Pasteur (28 rue du Docteur Roux, F-75024 Paris cedex 15) on 30/06/92, 12/01/99, 15/04/99, 15/04/99 and 07/06/05 under the following designations CNCM I-1225, CNCM I-2116, CNCM I-2168, CNCM I-2170 and CNCM I-3446, and the species Bifidobacterium longum (BB536). The strain of Bifidobacterium lactis CNCM I-3446 may be obtained from Hansen (Chr. Hansen A/S, 10-12 Boege Alle, P.O. Box 407, DK-2970 Hoersholm, Denmark).

According to one particular embodiment of the invention, the composition comprises at least two different microorganisms, in particular probiotic microorganisms, and/or metabolites and/or fractions thereof. These microorganisms may differ in terms of their nature, for example bacterium and fungus, alternatively in terms of their family, their genus or their species, or solely in terms of their strain.

The prebiotics suitable for the invention may be chosen from oligosaccharides produced from glucose, galactose, xylose, maltose, sucrose, lactose, starch, xylan, hemicellulose, inulin, gums, for example of acacia type, or a mixture thereof. More particularly, the oligosaccharide comprises at least one fructo-oligosaccharide. More particularly, this prebiotic may comprise a mixture of fructo-oligosaccharide and of inulin.

In one particular embodiment, the combination of active agents does not comprise a probiotic microorganism and/or a prebiotic.

Cosmetic Composition Intended for Oral Administration and Kits

According to one aspect of the invention, a combination of active agents in accordance with the invention may be used in a cosmetic composition suitable for oral administration.

A subject of the present invention is particularly a cosmetic composition suitable for oral administration, comprising a combination of active agents as defined above.

The term “cosmetic composition” is intended to mean, for example, a nutritional composition, for example a food supplement, comprising at least one combination of active agents in accordance with the invention.

The expressions “nutritional composition”, “nutraceutical composition” or “cosmeceutical composition” are synonyms herein.

The term “food supplement” is intended to denote herein a food product, the purpose of which is to supplement the normal diet and which constitutes a concentrated source of nutrients, i.e. vitamins and/or minerals, and/or other substances having a nutritional or physiological effect, alone or combined, sold in dose form, namely presentation forms such as gel capsules, lozenges, tablets, soft capsules, pills and other similar forms, and also sachets of powder to be diluted, ampoules of liquid, bottles equipped with a dropper and the other analogous forms of liquid preparations, or in the form of drinks.

A preferred cosmetic composition according to the invention is a food supplement.

A preferred cosmetic composition according to the invention, intended for oral administration, comprises:
at least one polyunsaturated fatty acid, the total content of polyunsaturated fatty acid being between 1% and 80% by weight, preferably between 5% and 70% by weight, more preferentially between 10% and 60% by weight and even more preferentially between 20% and 50% by weight,
at least one carotenoid, the total content of carotenoid being between 0.01% and 6% by weight, preferably between 0.02% and 4% by weight, more preferentially between 0.05% and 3% by weight and even more preferentially between 0.07% and 2% by weight,
optionally vitamin E in a content of between 0.001% and 10% by weight, preferably between 0.01% and 10% by weight, more preferentially between 0.1% and 5% by weight and even more preferentially between 0.2% and 2% by weight,
optionally vitamin C in a content of between 0.001% and 30% by weight, preferably between 0.01% and 25% by weight, more preferentially between 0.1% and 20% by weight and even more preferentially between 1% and 15% by weight, and
the percentages being given by weight relative to the total weight of the cosmetic composition.

According to a particular embodiment, the preferred cosmetic composition as defined above does not comprise any other active ingredient than at least one polyunsaturated fatty acid, at least one carotenoid, vitamin E and vitamin C.

Such a cosmetic composition suitable for oral administration, preferably a food supplement, can have the following contents:

| | % by weight relative to the total |
|---|---|
| Active agent | weight of the composition |
| Polyunsaturated fatty acid(s) | 35 to 37 |
| | (including omega |
| | 3 and omega 6) |
| Vitamin C | 2.4 to 2.5 |
| Vitamin E | 0.4 to 0.5 |
| Carotenoid(s) (preferably | 0.09 to 0.10 |
| lycopene) | |

A composition in accordance with the invention comprises a physiologically or cosmetically acceptable vehicle.

A combination of active agents and a composition in accordance with the invention make it possible, by virtue of their oral administration, to improve hair quality.

The invention does not relate to the therapeutic field.

The combination of active agents in accordance with the invention or the composition in accordance with the invention is administered orally.

The combinations of active agents and compositions in accordance with the invention, intended for oral administration, can in particular comprise all or only a part of the daily dose.

In the case of compositions suitable for oral administration, the use of an ingestible support is preferred. The ingestible support may be of diverse nature according to the type of composition under consideration.

For ingestion, numerous embodiments of oral compositions and in particular of food supplements are possible.

The formulation of such compositions may be carried out via any usual process known to those skilled in the art.

Thus, a competition in accordance with the invention can preferably take the form of a sugar-coated tablet, a gel capsule, a suspension, a gel, an emulsion, an oral solution, a tablet to be swallowed or chewed, a capsule, in particular a soft or hard capsule, a granule to be dissolved, a syrup, a lozenge or an oral ampoule.

It can preferably be in the form of a soft or hard capsule, preferably in the form of a soft capsule.

In particular, a combination of active agents in accordance with the invention can be used in any of the forms of food supplements or compacted or non-compacted powders. The powders may be diluted with water or in a fizzy drink. According to one preferred embodiment, an orally administered composition in accordance with the invention may be formulated in the form of a sugar-coated tablet, a gel capsule, a gel, an emulsion, a tablet, a capsule, a hydrogel, a compacted or non-compacted powder, or a liquid suspension or solution.

The oral compositions may be either in anhydrous form or in aqueous form.

A combination of active agents in accordance with the invention may be formulated with the usual excipients for such oral compositions, such as food supplements, namely in particular fatty and/or aqueous components, humectants, thickeners, preserving agents, texturing agents, taste agents and/or coating agents, antioxidants, preservatives and dyes that are common in the food sector.

Needless to say, those skilled in the art will take care to select the optional excipients and formulating agents and/or the amount thereof such that the advantageous properties of the combination according to the invention or of the composition comprising the combination according to the invention are not, or are not substantially, adversely affected by the envisaged addition.

The formulating agents and excipients for an oral composition, and in particular for food supplements, are known in this field and are not the subject of a detailed description herein.

The invention also relates to a cosmetic combination product or kit for use simultaneously, separately or spread out over time, preferably a food supplement, comprising a first composition comprising a part of the compounds forming the combination of active agents in accordance with the invention, and a second composition comprising at least the other part of the compounds forming said combination of active agents.

This supplement may be formulated in such a way that the two compositions are in the same forms or in different forms, for example chosen from those mentioned above. Such a kit may in particular be provided in one and the same packaging.

Cosmetic Use of the Combination of Main Active Agents or of a Composition Comprising them

A subject of the present invention is particularly the oral cosmetic use of a combination of active agents as defined above, comprising at least one polyunsaturated fatty acid and at least one carotenoid, for improving hair quality and more particularly hair quality at the level of a zone having grown before said use.

According to a particular embodiment, the present invention relates to an oral cosmetic use of a combination of active agents as defined above, comprising at least one polyunsaturated fatty acid and at least one carotenoid, for improving hair quality and more particularly hair quality at the level of a zone having grown before said use, the improvement of hair quality consisting in an increased fibre diameter and/or an increased fibre density and/or an improved quality of the hair fibre.

According to a further embodiment, the present invention relates to an oral cosmetic use of one, two or three polyunsaturated fatty acids, preferably one or two polyunsaturated fatty acids, associated with one carotenoid, and optionally vitamin E and/or vitamin C for improving hair quality and more particularly hair quality at the level of a zone having grown before said use, the improvement of hair quality consisting in an increased fibre diameter and/or an increased fibre density and/or an improved quality of the hair fibre.

According to a even more particular embodiment, the present invention relates to an oral cosmetic use of fish oil and blackcurrant seed oil associated with lycopene, preferably under the form of tomato extract, and optionally vitamin E and/or vitamin C for improving hair quality and more particularly hair quality at the level of a zone having grown before said use, the improvement of hair quality consisting in an increased fibre diameter and/or an increased fibre density and/or an improved quality of the hair fibre, and more preferably in an increased fibre diameter and/or an increased fibre density.

In particular, the present invention relates to the use as defined above, in which said at least one polyunsaturated fatty acid is used in the form of at least one oil chosen from evening primrose oil, borage oil, blackcurrant seed oil, walnut oil, soya bean oil, fish oil, sunflower oil, wheat germ oil, hemp oil, fenugreek oil, Echium oil, argan oil, rice bran oil, sesame oil, almond oil, hazelnut oil, chia oil, linseed oil, olive oil, avocado oil, safflower oil, camelina oil, rasberry seed oil, cranberry seed oil, blueberry seed oil, Rubus chamaemorus Linn. oil, sea buckthorn oil, cumin oil, kiwi seed oil, coriander oil, microalgal extract oil, zooplankton extract oil, crustacean and/or mollusc extract oil, or combinations thereof.

In particular, the present invention relates to the use as defined above, in which said at least one polyunsaturated fatty acid is an omega 3 and/or an omega 6.

Preferably, the omega 3 is chosen from 5,8,11,14,17-eicosapentaenoic acid, 4,7,10,13,16,19-docosahexaenoic acid, α-linolenic acid and stearic acid, or a mixture thereof.

Preferably, the omega 6 is chosen from γ-linolenic acid and/or linolenic acid.

In one advantageous embodiment, the present invention relates to the use as defined above, in which said at least one polyunsaturated fatty acid is used in the form of a blackcurrant seed oil and/or of a fish oil and/or said carotenoid is lycopene.

In one particularly advantageous embodiment, the present invention relates to the use as defined above, characterized in that the combination of active agents comprises, by way of additional active agent, at least one compound chosen from a vitamin or combinations thereof and preferably vitamin C and/or vitamin E.

A subject of the invention is particularly the use as defined above, in which the combination of active agents is used in a cosmetic composition suitable for oral administration.

The contents are variable according to the form of the cosmetic composition in which the combination of active agents in accordance with the invention is used.

The contents in the cosmetic composition, the form of the cosmetic composition and the daily doses administered are in particular as defined above.

It is understood in the context of the present invention that “the oral cosmetic use” covers the use of compositions administered orally, these compositions being, for example, in the form of a food supplement. These compositions produce an aesthetic and comfort effect on the hair, or alternatively an effect which has a beauty purpose, for example with a view to protecting it, keeping it in good condition, and in particular making it more attractive, by improving the hair quality.

Process

The invention also relates to a cosmetic process for improving hair quality and more particularly hair quality at the level of a zone having grown before the implementation of said process, comprising the oral administration, to an individual, of a combination of active agents, as defined above, of a cosmetic composition intended for oral administration as defined above, or of a combination product or kit as defined above.

According to a particular embodiment, the invention also relates to a cosmetic process for improving hair quality and more particularly hair quality at the level of a zone having grown before the implementation of said process, comprising the oral administration, to an individual, of a combination of active agents, as defined above, of a cosmetic composition intended for oral administration as defined above, or of a combination product or kit as defined above, the improvement of hair quality consisting in an increased fibre diameter and/or an increased fibre density and/or an improved quality of the hair fibre.

The processes according to the invention have the characteristics of cosmetic processes insofar as, in particular, they make it possible to improve the aesthetics of the hair. In addition, a combination of active agents or a composition such as, for example, a food supplement according to the invention can be used daily for several months, without a medical prescription. The present invention therefore clearly lies outside the therapeutic field.

Advantageously, the application of a process of the invention confers the advantages previously indicated as being associated with the use of a combination of active agents or of a composition in accordance with the invention, and can in particular increase the fibre diameter and/or increase the fibre density and/or increase hair volume and/or improve the quality of the hair fibre.

A cosmetic process according to the invention may be carried out in particular by administration of a cosmetic composition as defined above.

A process of the invention may be carried out on a daily basis for example, at a rate of, for example, a single administration per day or one administration twice a day, for example once in the morning and once in the evening, or three times a day, in particular at each meal.

A cosmetic process according to the invention may be carried out, for example, by daily administration of a composition formulated, for example, in the form of a sugar-coated tablet, a gel capsule, a suspension, a gel, an emulsion, an oral solution, a tablet to be swallowed or chewed, a capsule, in particular a soft or hard capsule, a granule to be dissolved, a syrup, a lozenge or an oral ampoule, in appropriate amount and number, depending on their form.

An effective amount of a combination of active agents in accordance with the invention may be administered in a single dose per day or in doses fractionated over the course of the day, for example two or three times per day.

A process according to the invention may advantageously comprise a single administration. A cosmetic process may be carried out over a time period ranging from one week to several weeks, or even several months, this period moreover possibly being repeated after periods without treatment, for several months or even several years.

By way of example, the administration of a combination of active agents in accordance with the invention can be carried out at a rate of twice a day, generally over a prolonged period of at least four weeks, or even four to fifteen weeks, comprising or not comprising one or more periods of interruption, or being repeated after a period of interruption.

The ingredients are mixed, before being formed, in the order and under conditions that are easily determined by those skilled in the art.

Thus, the cosmetic uses and/or processes according to the invention make it possible to improve hair quality, in particular in men or women.

In the description and the examples that follow, the ranges of values written in the form “between . . . and . . . ” include the stated lower and upper limits.

Other characteristics and advantages of the invention will emerge more clearly from the examples that follow, which are given as non-limiting illustrations.

### EXAMPLES

### Example 1

### Oral Composition in Soft Capsule Form

The oral composition has the formula given in the following table:

| | | Percentage of the active agent |
|---|---|---|
| | Amount of | (in weight relative to the total |
| | ingredient | weight of the composition |
| Ingredients | (in mg/capsule) | excluding the capsule) |
| Fish oil | 230.00 | 12.62 |
| | | (polyunsaturated fatty acids) |
| Blackcurrant seed oil | 230.00 | 23.98 |
| | | (polyunsaturated fatty acids) |
| Vitamin E | 4.10 | 0.43 |
| Vitamin C | 19.50 | 2.46 |
| Tomato extract | 5.50 | 0.097 |
| | | (lycopene) |
| Excipients: | | |
| Glyceryl monostearate | 30.03 | |
| Beeswax | 10.00 | |
| Soya bean lecithin | 10.00 | |

Calculation of the fatty acid distribution of the oils used:

(1) Fish oil:

One soft capsule provides 71.3 mg of omega 3, including:

34.5 mg of EPA (5,8,11,14,17-eicosapentaenoic acid) (omega 3),

20.7 mg of DHA (4,7,10,13,16,19-docosahexaenoic acid) (omega 3).

(2) Blackcurrant seed oil:

One soft capsule provides 135.47 mg of polyunsaturated fatty acids, including:

80.73 mg of LA (linolenic acid) (omega 6),

26.91 mg of GLA (γ-linolenic acid) (omega 6),

22.77 mg of ALA (α-linolenic acid) (omega 3),

5.06 mg of SDA (stearidonic acid) (omega 3).

The soft capsule has, for example, the following formula:

Capsule

| | |
|---|---|
| Fish gelatin | 127.776 |
| Glycerol | 56.893 |
| Iron oxide | 2.051 |
| Titanium dioxide | 2.921 |
| Purified water | 21.082 |

A soft capsule according to the invention can be prepared in the following way.

The fish oil, the blackcurrant seed oil, the vitamin E, the vitamin C, the tomato extract and the excipients are mixed in the presence of nitrogen. The mixture is then homogenized, then encapsulated in the soft capsule consisting of fish gelatin, glycerol, iron oxide, titanium dioxide and purified water.

The dosage is, for example, from 1 to 4 soft capsules per day, preferably 2 capsules/day.

This composition makes it possible in particular:

to limit hair loss, in particular by promoting anchoring of the hair,

to improve the growth of the hair fibre, in particular by contributing to the normal growth of the hair and by promoting hair thickness, in particular through the growth of thick hairs,

to improve hair volume, in particular by promoting hair abundance, by increasing hair mass and hair density, by increasing hair diameter, and by preventing and/or limiting the formation of thin hairs.

### Example 2

### Clinical Results

One hundred and eighteen women participated in this study and were randomized in two groups: a group receiving the nutritional supplement (79 subjects, including 40 menopausal women) and a control group without nutritional supplement (39 subjects, including 19 menopausal women).

These women were 18 to 65 years old, had a hair loss severity score corresponding to grade I according to the Ludwig scale and complained of a loss of volume.

Women with short hair (<5 cm) and with an amount of hairs in the telogen phase >30% were not included in the cohort.

The women of the supplemented group ingested two soft capsules as defined in example 1 per day, for 6 months.

The thickness of a lock of hair predefined at the start of the study was evaluated at TO, T3 months and T6 months at a distance of 10 cm from the scalp, i.e. at the level of a zone having grown before the taking of the food supplement. Since this lock was identified with a clip used as hair extensions, it was therefore always the same lock that was measured throughout the study.

This measurement was carried out by means of a marketed and patented apparatus, the Trichometer [www.haircheck.com; Cohen B. The cross section trichometer: a new device for measuring hair quantity, hair loss and hair growth, Dermatol Surg, 2008; 34: 900-911]. This technology is based on the fact that the hair density and diameter determines the hair mass, and that the measurement of the cross section of a lock of hair reflects the hair density and diameter.

The measurement of the cross section is expressed according to an index or “Trichometer index” (mm² of hair/cm² of skin×100). The more the “Trichometer index” increases, the more the hair mass increases.

The changes in the “Trichometer index” in the supplemented group and the control group are reported in Table X.

TABLE X
Changes in the “Trichometer index” at a distance of 10 cm from the scalp

| | | | | Intra-group comparison | |
|---|---|---|---|---|---|
| | | | | (p-values) | |
| | T0 | T3 | T6 | T3/T0 | T6/T0 |
| COMPOSITION | 59.20 ± 15.93 | 61.16 ± 16.49 | 63.10 ± 16.23 | S, p < 0.001 | S, p < 0.001 |
| AS DESCRIBED | | | | | |
| IN EXAMPLE 1 | | | | | |
| CONTROL | 63.13 ± 15.81 | 62.15 ± 16.08 | 61.21 ± 15.87 | NS, p = 0.359 | NS, |

| | Inter-group comparison | Comparison of changes | |
|---|---|---|---|
| | (p-values) | (p-values) | |
| COMPOSITION | NS, p = 0.208 | S, p = 0.008 | S, p < 0.001 |
| AS DESCRIBED | | | |
| IN EXAMPLE 1 vs | | | |
| CONTROL | | | |

The “Trichometer index” values do not vary in the control group without supplementation. On the other hand, entirely unexpectedly, the “Trichometer index” increases by 3.3% and by 6.6% after taking the food supplement for 3 and 6 months, respectively. These increases are statistically significant.

In other words, taking the food supplement for a minimum of three months makes it possible to increase the hair mass, at the level of hairs having already grown before the taking of the food supplement.

1-15. (canceled)

16. An oral cosmetic method for improving hair quality and more particularly hair quality at the level of a zone having grown before said administration of a combination of said active agents, comprising at least a step of administration of a cosmetic composition comprising a combination of active agents, comprising at least one polyunsaturated fatty acid and at least one carotenoid.

17. The oral cosmetic method according to claim 16, in which said at least one polyunsaturated fatty acid is used in the form of at least one oil chosen from evening primrose oil, borage oil, blackcurrant seed oil, walnut oil, soya bean oil, fish oil, sunflower oil, wheat germ oil, hemp oil, fenugreek oil, Echium oil, argan oil, rice bran oil, sesame oil, almond oil, hazelnut oil, chia oil, linseed oil, olive oil, avocado oil, safflower oil, camelina oil, rasberry seed oil, cranberry seed oil, blueberry seed oil, Rubus chamaemorus Linn. oil, sea buckthorn oil, cumin oil, kiwi seed oil, coriander oil, microalgal extract oil, zooplankton extract oil, crustacean and/or mollusc extract oil, or combinations thereof.

18. The oral cosmetic method according to claim 16, wherein said at least one polyunsaturated fatty acid is used in the form of a blackcurrant seed oil and/or of a fish oil.

19. The oral cosmetic method according to claim 16, wherein said at least one polyunsaturated fatty acid is an omega 3 and/or an omega 6.

20. The oral cosmetic method according to claim 16, wherein said at least one carotenoid is lycopene.

21. The oral cosmetic method according to claim 16, wherein said at least one carotenoid in the form of a lycopene-rich tomato extract.

22. The oral cosmetic method according to claim 16, wherein that the combination of active agents comprises, by way of additional active agent, vitamins.

23. The oral cosmetic method according to claim 16, wherein the vitamins are vitamin C and/or vitamin E.

24. The oral cosmetic method according to claim 16, wherein the combination of active agents is used in a cosmetic composition suitable for oral administration.

25. The oral cosmetic method according to claim 16, wherein said composition is in the form of a sugar-coated tablet, a gel capsule, a suspension, a gel, an emulsion, an oral solution, a tablet to be swallowed or chewed, a capsule, a granule to be dissolved, a syrup, a lozenge or an oral ampoule.

26. The oral cosmetic method according to claim 16, wherein said at least one polyunsaturated fatty acid is present in a content of between 1% and 80% by weight, relative to the total weight of the cosmetic composition.

27. The oral cosmetic method according to claim 16, wherein the carotenoid is present in a content of between 0.01% and 6% by weight, relative to the total weight of the cosmetic composition.

28. The oral cosmetic method according to claim 16, wherein the improvement of hair quality comprises an increased fibre diameter and/or an increased fibre density and/or an increased hair volume and/or an improved quality of the hair fibre.

29. Cosmetic composition intended for oral administration, comprising:
at least one polyunsaturated fatty acid, the total content of polyunsaturated fatty acid being between 1% and 80%,
at least one carotenoid, the total content of carotenoid being between 0.01% and 6%,
optionally vitamin E in a content of between 0.001% and 10%, and
optionally vitamin C in a content of between 0.001% and 30%,
the percentages being given by weight relative to the total weight of the cosmetic composition.

30. Cosmetic combination product or kit for use simultaneously, separately or spread out over time, comprising a first cosmetic composition comprising a part of the compounds forming the combination of active agents comprising at least one polyunsaturated fatty acid and at least one carotenoid and a second cosmetic composition comprising at least the other part of the compounds forming said combination of active agents.
